(19) United States
(12) Patent Application Publication
(10) Pub. No.: US 2018/0348194 A1
(43) Pub. Date: Dec. 6, 2018

(54) METHOD OF ANALYZING A WET BLOOD SAMPLE

(71) Applicant: Thermo Finnigan LLC, San Jose, CA (US)

(72) Inventors: Cornelia L. BOESER, San Jose, CA (US); John GLAZIER, San Jose, CA (US); Marta KOZAK, Palo Alto, CA (US)

(21) Appl. No.: 15/609,606

(22) Filed: May 31, 2017

(51) Int. Cl. G01N 33/49; G01N 33/487

(52) U.S. Cl. CPC G01N 33/49; H01J 49/0031; G01N 33/4875

(57) ABSTRACT

A method of analyzing a wet blood sample is disclosed. The method includes dispensing a wet blood sample onto a substrate. The method also includes spotting a zinc sulfate solution onto the wet blood sample to fix or set the wet blood sample in place on the substrate, thereby trapping blood components inside the blood spot. The method further includes generating ions of an analyte in the wet blood sample and analyzing the ions.

### FIELD OF THE INVENTION

This invention relates to analysis of whole blood. More specifically, this invention relates to analysis of wet blood spots by using a zinc sulfate solution to fix the blood in place, while analytes in the blood are eluted for subsequent quantitative analysis.

### BACKGROUND OF THE INVENTION

For quantitative analysis of whole blood spotted on a substrate, blood spots are traditionally ‘thoroughly dried’ before the analysis. To achieve ‘thorough drying’, the sample must be either dried at room temperature for at least 90 minutes or at elevated temperatures of 40-50 C for at least 25 minutes. When spray solvent is applied and moves through dried blood towards the tip of the substrate it extracts analytes which are ionized to produce a plurality of ions detectable in a mass spectrometer. The blood spot itself stays intact and in place—drying thus ‘fixes’ the blood onto the substrate, but is time-consuming. Alternatively, if wet blood is used instead of dried blood and comes in contact with the spray solvent, it moves toward the tip of the substrate and eventually is sprayed off the substrate into the mass spectrometer, as seen in FIG. 2A which shows 5 μL fresh blood spotted onto a paper substrate contained in a disposable cartridge, with 100 μL spray solvent (methanol) applied to the cartridge. The blood spraying off the substrate contaminates the instrument and causes unwanted interferences from blood components with the measurement of target analytes.

What is needed is a method of analyzing wet blood samples that eliminates the time-consuming nature of drying, prevents contamination of the instrument capable of performing mass analysis, and is without the unwanted interferences from blood components with the measurement of target analytes.

### SUMMARY

Embodiments of the present invention disclose methods and systems for analysis of wet blood samples. In one embodiment, a method of analyzing a wet blood sample is disclosed. The method includes dispensing a wet blood sample onto a substrate. The method further includes spotting a zinc sulfate solution onto the wet blood sample to fix or set the wet blood sample in place on the substrate, thereby trapping blood components inside the blood spot. The method also includes generating ions of an analyte in the wet blood sample and analyzing the ions. It should be noted that the zinc sulfate solution and the wet blood sample can be dispensed simultaneously, sequentially, or separately, in any order, onto the substrate prior to ionization and mass analysis of the analytes in the wet blood sample.

The substrate is preferably a porous substrate. The porous substrate may comprise, but is not limited to, a filter paper or a polymer material.

In one embodiment, the step of generating ions of an analyte in the wet blood sample comprises applying a solvent and voltage to the substrate to generate the ions.

The solvent may comprise, but is not limited to, an organic solvent, an aqueous solvent, or a mixture thereof.

In one embodiment, the step of analyzing the ions comprises providing a mass analyzer to generate a mass spectrum of the analyte. The mass analyzer may be enclosed within a mass spectrometer. The mass analyzer is, but not limited to, one of the following: a triple quadrupole analyzer, an ion trap analyzer, or an Orbitrap mass analyzer.

The analyte comprises a protein, a peptide, a metabolite, an endogenous hormone, a therapeutic drug, drugs of abuse, or combinations thereof.

In one embodiment, the whole blood sample and the zinc sulfate solution each has a volume of about 2 μL to about 15 μL, and the zinc sulfate solution comprises zinc sulfate present at a concentration of about 10 mM to about 100 mM in methanol.

In another embodiment of the present invention, a system for analyzing a wet blood sample is disclosed. The system includes a substrate onto which a wet blood sample and a zinc sulfate solution is dispensed. The zinc sulfate solution and the wet blood sample may be dispensed simultaneously, sequentially, or separately, in any order, onto the substrate. The system also includes an ionization source and a mass analyzer.

### BRIEF DESCRIPTION OF THE DRAWINGS

FIG. 1 is a flow chart depicting steps of a method of analyzing a wet blood sample, in accordance with one embodiment of the present invention.

FIG. 2A shows 5 μL fresh blood spotted onto a paper substrate contained in a disposable cartridge, with 100 μL spray solvent (methanol) applied to the cartridge.

FIG. 2B shows 5 μL of fresh blood spotted onto a paper substrate contained in a disposable cartridge, with a 5 μL 30 mM ZnSO₄ solution in methanol spotted onto the blood and 100 μL spray solvent (methanol) applied to the cartridge.

FIG. 2C shows 5 μL fresh blood spotted onto a paper substrate, with 5 μL methanol spotted onto the blood and 100 μL spray solvent (methanol) applied to the cartridge.

FIG. 3A shows the effects of different concentrations and volumes of ZnSO4 in methanol on the integrity of a blood spot.

FIG. 3B shows the effects of different concentrations and volumes of ZnSO4 in methanol on the integrity of a blood spot.

FIG. 4 is a graph comparing the amount of analyte (Cyclosporin A) extracted from a wet blood spot: a) with only solvent solution applied (left bar) and b) with application of both solvent solution and ZnSO₄ solution (right bar).

FIG. 5 shows a calibration curve for quantitation of Cyclosporin A covering extended concentration range.

FIG. 6A shows 1 mL whole blood, re-suspended in plasma after a centrifuge and removal process, spotted onto a paper substrate contained in a disposable cartridge, with 50 mM of ZnSO₄ in methanol spotted onto the blood.

FIG. 6B shows 1 mL whole blood, re-suspended in a protein standard after a centrifuge and removal process, spotted onto a paper substrate contained in a disposable cartridge, with 50 mM of ZnSO₄ in methanol spotted onto the blood.

### DETAILED DESCRIPTION OF EMBODIMENTS

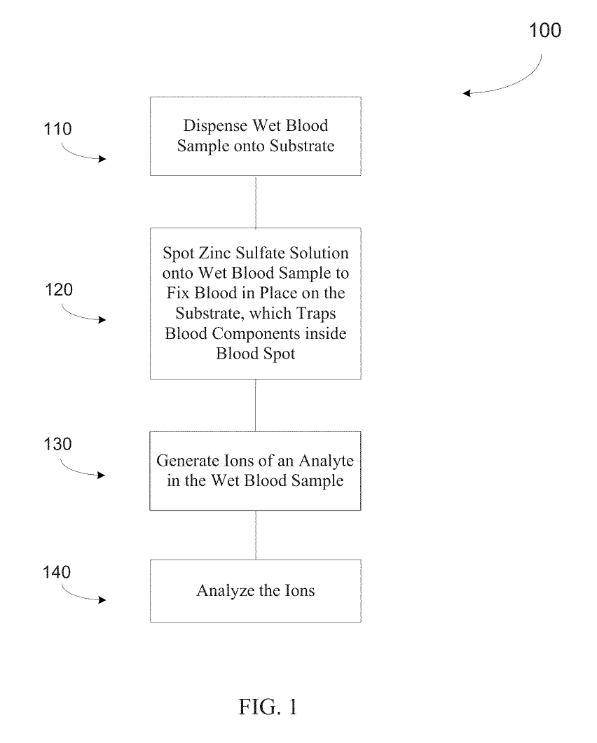

FIG. 1 is a flow chart 100 depicting steps of a method of analyzing an analyte contained in a wet blood sample, in accordance with one embodiment of the present invention. The analyte may comprise, in various implementations, a protein, a peptide, a therapeutic drug or its metabolite, a drug of abuse or its metabolites, an endogenous substance such as creatinine, or a combination thereof. In step 110, a wet whole blood sample is dispensed (e.g., spotted) onto a substrate. The substrate is preferably a porous substrate such as, but not limited to, paper. In particular embodiments, the porous material is filter paper. Exemplary filter papers include cellulose filter paper, ashless filter paper, nitrocellulose paper, glass microfiber filter paper, and polyethylene paper. Filter paper having any pore size may be used. Exemplary pore sizes include Grade 1 (11 μm), Grade 2 (8 μm), Grade 595 (4-7 μm), and Grade 6 (3 μm). The wet blood sample is collected and dispensed by any method known in the art. For example, the blood sample can be aspirated by drawing blood from a blood sample tube into a syringe or a pipette and dispensing on to the substrate. The pipette or syringe is capable of dispensing and/or aspirating about 0.5 microliters (“μL”) to about 5 milliliters (“mL”) of blood.

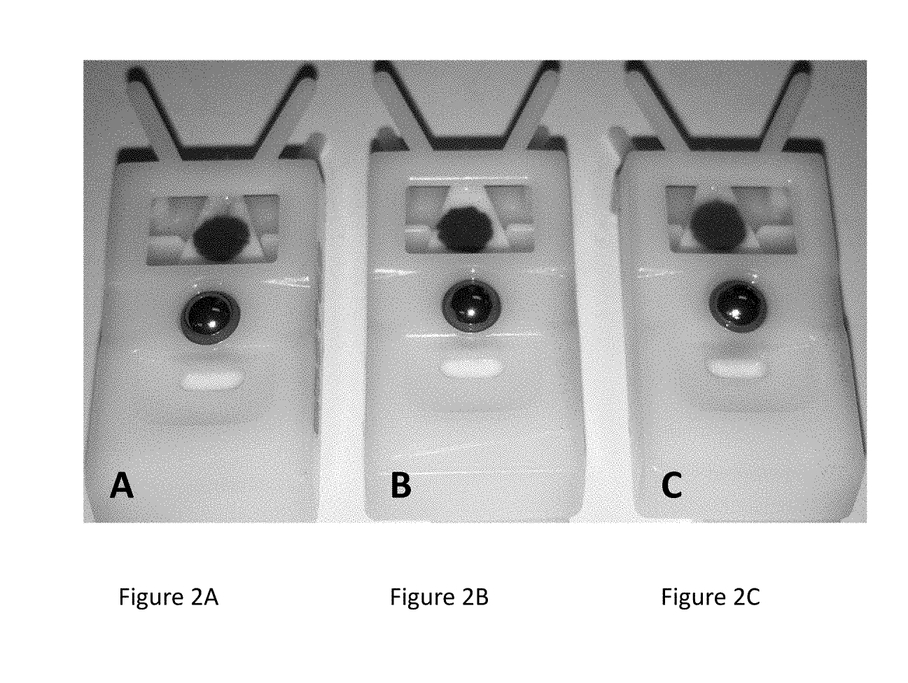

Next, in step 120, a roughly equivalent amount of zinc sulfate solution is spotted or dispensed onto the wet blood sample on the substrate. In one embodiment, the zinc sulfate solution comprises zinc sulfate present at a concentration of about 10 mM to about 100 mM in methanol. The zinc sulfate solution fixes the blood onto the substrate and partially precipitates proteins from the blood sample, as shown in FIG. 2B, trapping heavy blood components inside the blood spot. FIG. 2B shows 5 μL of fresh blood spotted onto a paper substrate contained in a disposable cartridge, with a 5 μL 30 mM ZnSO₄ solution in methanol spotted onto the blood and 100 μL spray solvent (methanol) applied to the cartridge. In this embodiment, where the substrate tapers to a tip, the zinc sulfate solution prevents blood from moving to the tip. This is in contrast to the control experiment of FIG. 2C where 5 μL methanol was spotted directly on top of the blood spot before application of 100 μL methanol and without any zinc sulfate solution. It should be noted that in the example of FIG. 2B, the zinc sulfate solution is dispensed after the blood is spotted onto the substrate. However, the order of spotting or dispensing can be reversed in that the zinc sulfate solution precedes the blood on the substrate. In an alternative embodiment, the blood sample and the zinc sulfate solution may be dispensed simultaneously onto the substrate. It should also be noted that the substrate can take any number of geometries and does not need to taper to a tip.

Next, in step 130, analyte ions are generated by ionizing molecules in the wet blood sample using an appropriate ionization technique. In one embodiment, a voltage is applied to the substrate to generate ions of the protein or peptide in the blood sample that are expelled from the substrate. In one particular implementation, the ion source may take the form of a direct sampling ion source such as the Paper Spray ionization in which the blood sample is deposited on a porous wicking material (e.g., paper or polymer) and electrosprayed from a tip of the porous paper or polymer material.

Next, in step 140, the analyte ions are analyzed, thereby analyzing the analyte (e.g., protein or peptide) in the blood sample. In one embodiment, the analysis comprises of providing a mass analyzer to generate a mass spectrum of a protein, a peptide, a peptide, a metabolite, an endogenous hormone, a therapeutic drug, drugs of abuse, or combinations thereof. The mass analyzer can be, but is not limited to, an ion trap mass analyzer, a quadrupole ion trap, or an Orbitrap.

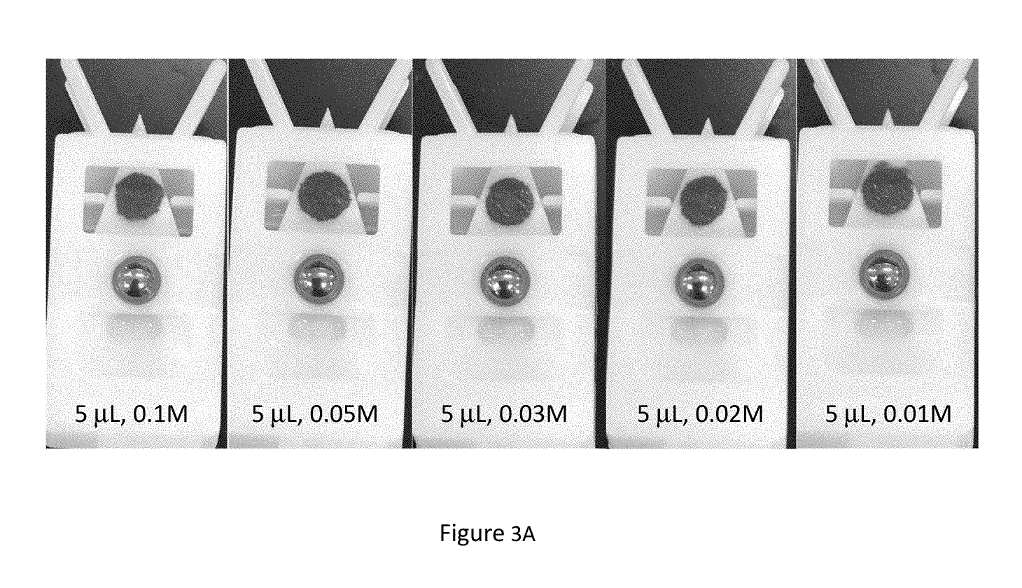

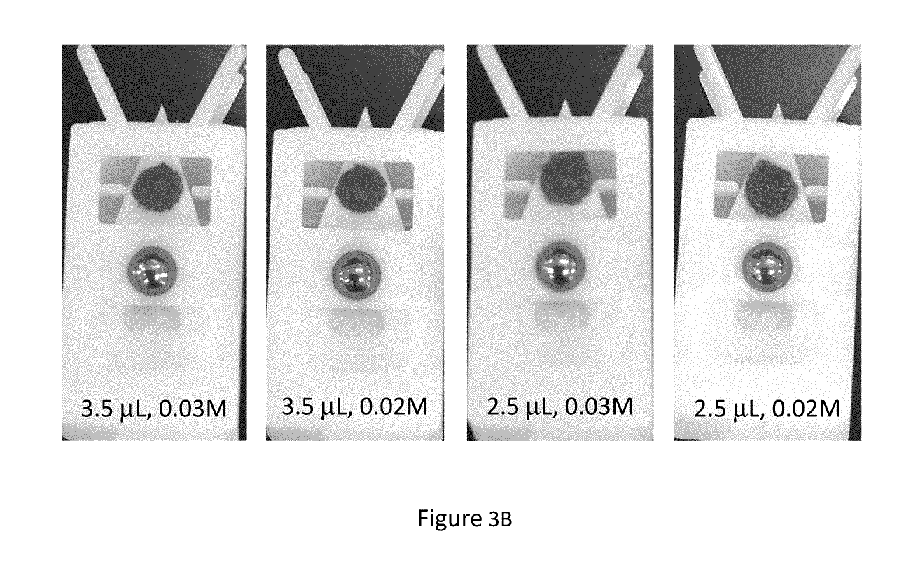

FIG. 3A and FIG. 3B show the effects of different concentrations and volumes of ZnSO₄ in methanol on the integrity of a wet blood spot. The blood sample volumes and ZnSO₄ concentrations in methanol were as follows in FIG. 3A (from left to right): 5 μL blood and 100 mM ZnSO₄ in methanol; 5 μL blood and 50 mM ZnSO₄ in methanol; 5 μL blood and 30 mM ZnSO₄ in methanol; 5 μL blood and 20 mM ZnSO₄ in methanol; and 5 μL blood and 10 mM ZnSO₄ in methanol. The blood sample volumes and ZnSO₄ concentrations in methanol were as follows in FIG. 3B (from left to right): 3.5 μL blood and 30 mM ZnSO₄ in methanol; 3.5 μL blood and 20 mM ZnSO₄ in methanol; 2.5 μL blood and 30 mM ZnSO₄ in methanol; and 2.5 μL blood and 20 mM ZnSO₄ in methanol. Based on these experimental results, a volume of 5 μL and concentration of 30 mM ZnSO₄ in methanol was experimentally established as optimum conditions to efficiently fix a whole blood sample onto a paper substrate. Lower concentrations and/or volume resulted in partial movement of blood components toward the paper, as seen in some of the samples in FIG. 3A and FIG. 3B.

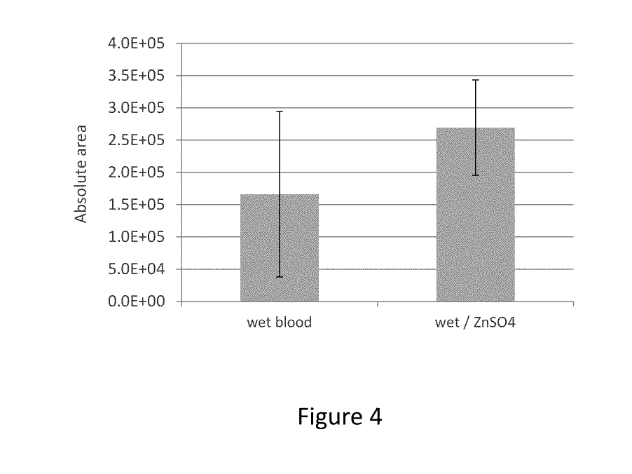

FIG. 4 is a graph comparing the amount of analyte (Cyclosporin A) extracted from a wet blood spot: a) with only solvent solution applied (left bar) and b) with application of both solvent solution and ZnSO₄ solution (right bar). 5 μL wet blood was used in both samples. For the sample with ZnSO₄, 30 mM ZnSO₄ in methanol was dispensed on top of the wet blood spot. In both samples, 100 μL methanol was used as the spray solvent, with an integration time of approximately 1 minute. The transition that was monitored was m/z 1224.8→m/z 1112.9. In other words, the parent ion had an m/z of 1224.8. The parent ion was fragmented in an MS/MS experiment, which generates multiple fragments with different m/z values. In the example of FIG. 4, only one of those fragments was monitored (the fragment ion with m/z 1112.9) and integrated over 1 minute. The integrated area of this particular fragment is shown on the Y-axis in FIG. 4. As shown in the figure, the application of ZnSO₄ to blood increases the amount of analyte extracted from the blood spot and decreases the % RSD (relative standard deviation).

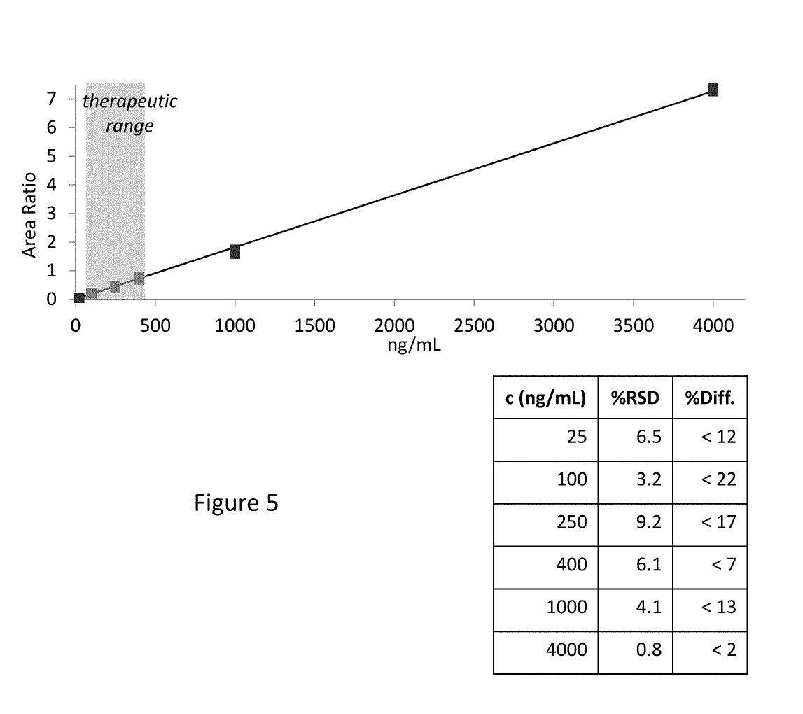

FIG. 5 shows a calibration curve for quantitation of Cyclosporin A of the measured area ratio versus concentration. The role of a calibration curve is to permit accurate measurement of the level of an analyte in a sample. To generate a calibration curve, a series of calibrator samples having increasing concentrations of an analyte, in this case the immunosuppressant Cyclosporin A, and a fixed concentration of an internal standard, in this case Cyclosporin D, are subjected to mass spectrometry where one or more mass spectrometry signals of the analyte and its internal standard are measured. Generally an internal standard is used when performing quantitation using a mass spectrometry technique. This standard serves as a control for loss of analyte during sample preparation and instrument injection, and ion variability. An internal standard useful in the methods described herein can be isotopically labeled. In some cases, test sample preparation can involve mixing the blood sample with an extraction solution (e.g., methanol) in which one or more internal standards have been added. Alternatively, the internal standards can be added to a mixture of the blood sample and an extraction solution at any step in the sample preparation that ensures the internal standards will not be removed from the mixture during the sample processing. The internal standard is generally added prior to sample preparation and analysis, and is added at the same level in every sample including the test sample.

Mass spectrometry is used to detect and measure the signal intensities (e.g., area) of the analyte and, if desired, area ratios of the analyte and an internal standard can be used to determine amount of the analyte in each test sample by relating an analyte/internal standard signal ratio from the test sample to the calibration curve. In the example of FIG. 5, the sample solution included 5 μL of blood containing the Cyclosporin A, Cyclosporin D as the internal standard, and 30 mM of ZnSO4 in methanol, fixed onto the paper substrate. The integration time was approximately 0.9-1.4 minutes. The table on the right of the figure shows precision and accuracy values for the data points referenced in the calibration curve in FIG. 5. Precision is expressed as % RSD of area ratios (analyte to internal standard). Accuracy is expressed as % difference in response compared to value determined by the calibration curve. Using the method described in connection with FIG. 1, excellent sensitivity and high quantitation precision have been obtained with wet blood samples of 5 μL.

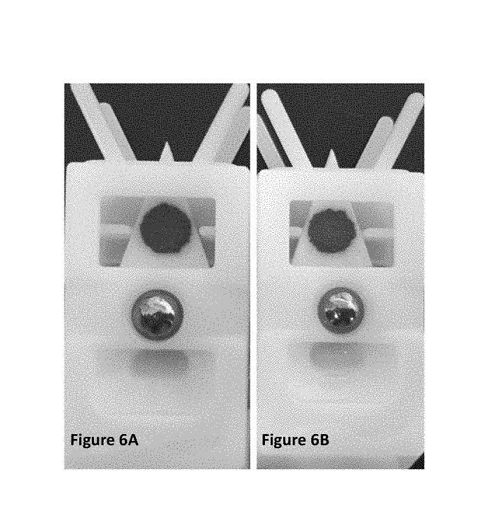

FIGS. 6A and 6B show another experiment where 1 mL of whole blood was spotted onto a paper substrate within a disposable cartridge. The sample in FIG. 6A was centrifuged, which separated red blood cells from plasma, and then the plasma was removed from the sample. The red blood cells were subsequently re-suspended in plasma, mixed together, and spotted onto the paper substrate within the cartridge. This sample served as a process control. A 50 mM ZnSO₄ solution in methanol was then spotted onto the blood. This causes precipitation of plasma proteins while the blood spot is fixed in place, as seen in FIG. 6A. The 1 mL whole blood sample in FIG. 6B was also centrifuged and plasma separated. However, the red blood cells were then re-suspended in a protein standard containing 80 mg/mL human serum albumin (HSA) and globulins. The protein standard contains protein in amounts comparable to human plasma. The image of FIG. 6B shows that the protein standard works as well as plasma to fix blood in place. This experiment proved that the mechanism of fixing blood in placing using ZnSO₄ is based on protein precipitation.

The present invention has been described in terms of specific embodiments incorporating details to facilitate the understanding of the principles of construction and operation of the invention. As such, references herein to specific embodiments and details thereof are not intended to limit the scope of the claims appended hereto. It will be apparent to those skilled in the art that modifications can be made in the embodiments chosen for illustration without departing from the spirit and scope of the invention.

What is claimed is:

1. A method of analyzing a wet blood sample comprising:
a. dispensing a wet blood sample onto a substrate;
b. spotting a zinc sulfate solution onto the wet blood sample to fix or set the wet blood sample in place on the substrate, thereby trapping blood components inside the blood spot;
c. generating ions of an analyte in the wet blood sample; and
d. analyzing the ions.

2. The method of claim 1 wherein the substrate comprises a porous substrate.

3. The method of claim 2 wherein the porous substrate comprises filter paper.

4. The method of claim 1 wherein the generating ions of an analyte in the wet blood sample comprises applying a solvent and voltage to the substrate to generate the ions.

5. The method of claim 4 wherein the solvent comprises an organic solvent, an aqueous solvent, or a mixture thereof.

6. The method of claim 1 wherein the analyzing the ions comprises providing a mass analyzer to generate a mass spectrum of the analyte.

7. The method of claim 6 wherein the mass analyzer is enclosed within a mass spectrometer.

8. The method of claim 6 wherein the mass analyzer is selected from the group consisting of: a triple quadrupole, an ion trap, or an Orbitrap.

9. The method of claim 1 wherein the analyte comprises a protein, a peptide, a metabolite, an endogenous hormone, a therapeutic drug, drugs of abuse, or combinations thereof.

10. The method of claim 1 wherein the whole blood sample and the zinc sulfate solution each has a volume of about 2 μL to about 15 μL, and the zinc sulfate solution comprises zinc sulfate present at a concentration of about 10 mM to about 100 mM in methanol.

11. A method of analyzing a wet blood sample comprising:
a. dispensing a wet blood sample and a zinc sulfate solution onto a substrate at substantially the same time;
b. generating ions of an analyte in the wet blood sample; and
c. analyzing the ions.

12. The method of claim 11 wherein the blood sample and the zinc sulfate solution are dispensed simultaneously, sequentially, or separately, in any order, onto the substrate.

13. The method of claim 12 wherein the zinc sulfate solution fixes or sets the wet blood sample in place on the substrate.

14. The method of claim 11 wherein the substrate comprises a porous substrate.

15. The method of claim 14 wherein the porous substrate comprises filter paper.

16. The method of claim 11 wherein the generating ions of an analyte in the wet blood sample comprises applying a solvent and voltage to the substrate to generate the ions.

17. The method of claim 16 wherein the solvent comprises an organic solvent, an aqueous solvent, or a mixture thereof.

18. The method of claim 11 wherein the analyzing the ions comprises providing a mass analyzer to generate a mass spectrum of the analyte.

19. The method of claim 18 wherein the mass analyzer is enclosed within a mass spectrometer.

20. The method of claim 18 wherein the mass analyzer is selected from the group consisting of: a triple quadrupole, an ion trap, or an Orbitrap.

21. The method of claim 11 wherein the analyte comprises a protein, a peptide, a metabolite, an endogenous hormone, a therapeutic drug, drugs of abuse, or combinations thereof.

22. The method of claim 11 wherein the wet blood sample and the zinc sulfate solution each has a volume of about 2 μL to about 15 μL, and the zinc sulfate solution comprises zinc sulfate present at a concentration of about 10 mM to about 100 mM in methanol.

23. A system for analyzing a wet blood sample comprising:
a. a substrate onto which has been dispensed a wet blood sample and a zinc sulfate solution, wherein the zinc sulfate solution and the wet blood sample are dispended simultaneously, sequentially, or separately, in any order, onto the substrate;
b. an ionization source; and
c. a mass analyzer.

24. The system of claim 23 wherein the substrate comprises a porous substrate.

25. The system of claim 23 wherein the porous substrate comprises filter paper.

26. The system of claim 23 further comprising a solvent applied to the substrate.

27. The system of claim 26 wherein the solvent comprises an organic solvent, an aqueous solvent, or a mixture thereof.

28. The system of claim 23 wherein the ionization source is configured to generate ions of an analyte in the wet blood sample.

29. The system of claim 28 wherein the mass analyzer is configured to generate a mass spectrum of the analyte.

30. The system of claim 29 wherein the mass analyzer is enclosed within a mass spectrometer.

31. The system of claim 29 wherein the mass analyzer is selected from the group consisting of: a triple quadrupole, an ion trap, or an Orbitrap.

32. The system of claim 23 wherein the wet blood sample and the zinc sulfate solution each has a volume of about 2 μL to about 15 μL, and the zinc sulfate solution comprises zinc sulfate present at a concentration of about 10 mM to about 100 mM in methanol.
